(19) United States
(12) Patent Application Publication
(10) Pub. No.: US 2014/0144432 A1
(43) Pub. Date: May 29, 2014

(54) ORAL SUCTION DEVICE WITH VENTILATION CAPABILITY

(71) Applicants: Rafi Avitsian, Solon, OH (US); Andrew Zura, Cleveland, OH (US)

(72) Inventors: Rafi Avitsian, Solon, OH (US); Andrew Zura, Cleveland, OH (US)

(73) Assignee: THE CLEVELAND CLINIC FOUNDATION, Cleveland, OH (US)

(21) Appl. No.: 13/878,952

(22) PCT Filed: Oct. 14, 2011
(86) PCT No.: PCT/US11/56378

§ 371 Date: May 16, 2013

Related U.S. Application Data
(60) Provisional application No. 61/393,239, filed on Oct. 14, 2010.

(51) Int. Cl. A61M 16/04; A61M 16/00

(52) U.S. Cl. CPC A61M 16/0463; A61M 16/0057
USPC 128/202.16

(57) ABSTRACT

An oral suction device for insertion into an airway of a patient including a first tube and a second tube. The first tube includes a first gas port configured to be coupled to a ventilation source. The second tube extends coaxially over at least a first section of the first tube and includes a second gas port configured to be coupled to a negative pressure source. The second tube also has an outer surface with at least one hole defined therein.

### CROSS-REFERENCE TO RELATED APPLICATIONS

This application claims priority under 35 USC §119(e) to U.S. Provisional Application Ser. No. 61/393,239, filed on Oct. 14, 2010, the entire disclosure of which is incorporated herein by reference.

### TECHNICAL FIELD

The present disclosure relates generally to medical instruments for use with a patient, and, more specifically, to an oral suction device configured to be partially inserted into a patient's airway.

### BACKGROUND

Oral suction is often performed to evacuate fluids and other materials from a patient's airway during medical procedures. Often oral suction devices include a rigid tubular member fluidly coupled to a negative pressure source. Oral suction devices are sometimes used in conjunction with other devices such as ventilation or intubation tubes.

### SUMMARY

The present application discloses one or more of the features recited in the appended claims and/or the following features which, alone or in any combination, may comprise patentable subject matter.

According to one aspect of the disclosure, an oral suction device for insertion into an airway of a patient is disclosed. The oral suction device includes a first tube and a second tube. The first tube includes a first gas port configured to be coupled to a ventilation source. The second tube extends coaxially over at least a first section of the first tube. The second tube includes a second gas port configured to be coupled to a negative pressure source and an outer surface having at least one hole defined therein.

In some embodiments, the second gas port may include a valve operable to fluidly couple the negative pressure source with the at least one hole. Additionally, in some embodiments, the outer surface of the second tube may include a top section and a bottom section. In such embodiments, the top surface section may have a first hole defined therein and the bottom surface section may have a second hole defined therein.

According to another aspect, the oral suction device includes a first tube and a second tube coupled to the first tube. The first tube includes a first gas port configured to be coupled to a ventilation source. The second tube defines a chamber, and a first section of the first tube is positioned in the chamber of the second tube. The second tube includes a second gas port configured to be coupled to a negative pressure source and at least one hole extending from the chamber to an outer surface of the second tube.

In some embodiments, the second gas port may include a valve. The valve may be operable to fluidly couple the negative pressure source with the chamber. In some embodiments, the second tube may include a sidewall defining the chamber, and the first section of the first tube may be spaced apart from the sidewall.

According to another aspect, the oral suction device includes a first tube and a second tube. The first tube having a first gas port configured to be coupled to a ventilation source. The second tube extends coaxially over at least a first section of the first tube. The second tube includes a second gas port and an outer surface having at least one hole defined therein. The second gas port is configured to be coupled to a negative pressure source.

In some embodiments, the second gas port may be located at a proximal end of the second tube. The outer surface of the second tube may include a top section having a first hole defined therein. The outer surface may include a bottom section opposite the top section. The bottom section may have a second hole defined therein. The bottom section of the outer surface may be configured to face the patient's tongue when the oral suction device is inserted into the airway of a patient. The second hole defined in the bottom section may be sized to permit air from the second tube to pull the patient's tongue into contact with the outer surface of the second tube when negative pressure is introduced into the second tube through the second gas port.

In some embodiments, the oral suction device may include an end piece extending outward from a proximal end of the second tube. The end piece may include a block member extending outwardly from the second tube, and a flange extending outwardly from a proximal end of the block member. It is contemplated that the oral suction device may include a hose coupled to the second gas port and extending outwardly from the block member. The flange of the end piece may extend outwardly from a proximal end of the first tube. The flange may be sized to prevent passage of the flange past the lips of the patient when the oral suction device is inserted in the airway of the patient.

In some embodiments, the oral suction device may include an end wall extending inwardly from a distal end of the second tube to the first tube. A second section of the first tube may extend outwardly from the end wall. The second section of the first tube may be tapered.

In some embodiments, the first tube and the second tube may be curved to conform to the airway of the patient when the oral suction device is inserted into the airway of the patient. The first tube may be a predetermined length such that a distal end of the first tube is positioned in a superglotic region of the airway of the patient when the oral suction device is inserted in the airway of the patient.

According to another aspect, the oral suction device includes a first tube and a second tube coupled to the first tube. The first tube including a first gas port configured to be coupled to a ventilation source. The second tube includes a chamber defined therein and a second gas port configured to be coupled to a negative pressure source. A first section of the first tube is positioned in the chamber of the second tube. The second tube includes at least one hole extending between the chamber and an outer surface of the second tube.

In some embodiments, a second section of the first tube may extend out of the chamber. The first section of the first tube may include a proximal end of the first tube and the second section of the first tube may include a distal end of the first tube. The distal end of the first tube may be tapered around a circumference of the first tube.

In some embodiments, the oral suction device may include an end wall extending between a distal end of the second tube and an outer surface of the first tube to enclose a distal end of the chamber. A distal end of the first tube may extend from the end wall.

In some embodiments, the oral suction device may include an end piece connecting the first tube and the second tube to enclose a proximal end of the chamber. The end piece may be configured to engage a patient's lips and may be sized to prevent the end piece from passing beyond the patient's lips when the oral suction device is inserted in the airway of the patient. The end piece may include a block member extending outwardly from the second tube, and a flange extending outwardly from a proximal end of the block member.

In some embodiments, the second tube may have an oblong cross section such that the chamber has an oblong cross section. The second tube may be curved such that a distal end of the chamber is angled approximately ninety degrees from a proximal end of the chamber. The first tube may be curved such that a distal end of the first tube is angled approximately ninety degrees from a proximal end of the first tube.

In some embodiments, the second tube may include a bottom section having at least one hole extending between the chamber and the outer surface of the second tube. The bottom section may be configured to face the patient's tongue when the oral suction device is inserted into the airway of the patient. The second tube may include a top section opposite the bottom section. The top section may have at least one hole extending between the chamber and the outer surface of the second tube. The second tube may include a side section connecting the bottom section and the top section. The side section may have at least one hole extending between the chamber and the outer surface of the second tube.

According to another aspect, a method of providing suction to an airway of a patient is disclosed. The method includes inserting an oral suction device into a patient's airway, applying ventilation to first tube of the oral suction device, and applying vacuum pressure to the second tube of the oral suction device. The oral suction device includes a first tube having a first gas port configured to be coupled to a ventilation source; and a second tube extending coaxially over at least a first section of the first tube, the second tube configured to be coupled to a negative pressure source.

In some embodiments, applying vacuum pressure to the second tube of the oral suction device may include drawing the patient's tongue into contact with the second tube by passing vacuum pressure through a first hole formed through a bottom side of the second tube. It is also contemplated that applying vacuum pressure to the second tube of the oral suction device may include removing unwanted materials from the patient's mouth by passing vacuum pressure through a second hole formed through a second side of the second tube.

In some embodiments, applying ventilation to the first tube of the oral suction device may include applying positive ventilation pressure to the first tube and applying negative ventilation pressure to the first tube. Applying vacuum pressure to the second tube may only be performed when negative ventilation pressure is applied to the first tube.

In some embodiments, inserting the oral suction device into a patient's airway may include positioning a block member between the patient's upper teeth and the patient's bottom teeth. The block member may extend outwardly from the second tube. It is contemplated that inserting the oral suction device into a patient's airway may include positioning a flange in contact with the patient's lips. The flange may extend outwardly from a proximal end of the block member and may be sized to discourage the flange from being passed beyond the patient's lips.

### BRIEF DESCRIPTION OF THE DRAWINGS

The detailed description particularly refers to the accompanying figures, in which:

FIG. 1 is a perspective view of one embodiment of an oral suction device;

FIG. 2 is a diagrammatic side view of the oral suction device of FIG. 1 inserted in a patient's airway;

FIG. 3A is a side view of another embodiment of the oral suction device;

FIG. 3B is a bottom view of the oral suction device of FIG. 3A;

FIG. 4A is a diagrammatic side view of oral suction device of FIGS. 3A and 3B illustrating air flow through the device; and

FIG. 4B is a diagrammatic bottom view of oral suction device of FIGS. 3A-4A illustrating air flow through the device.

### DETAILED DESCRIPTION

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

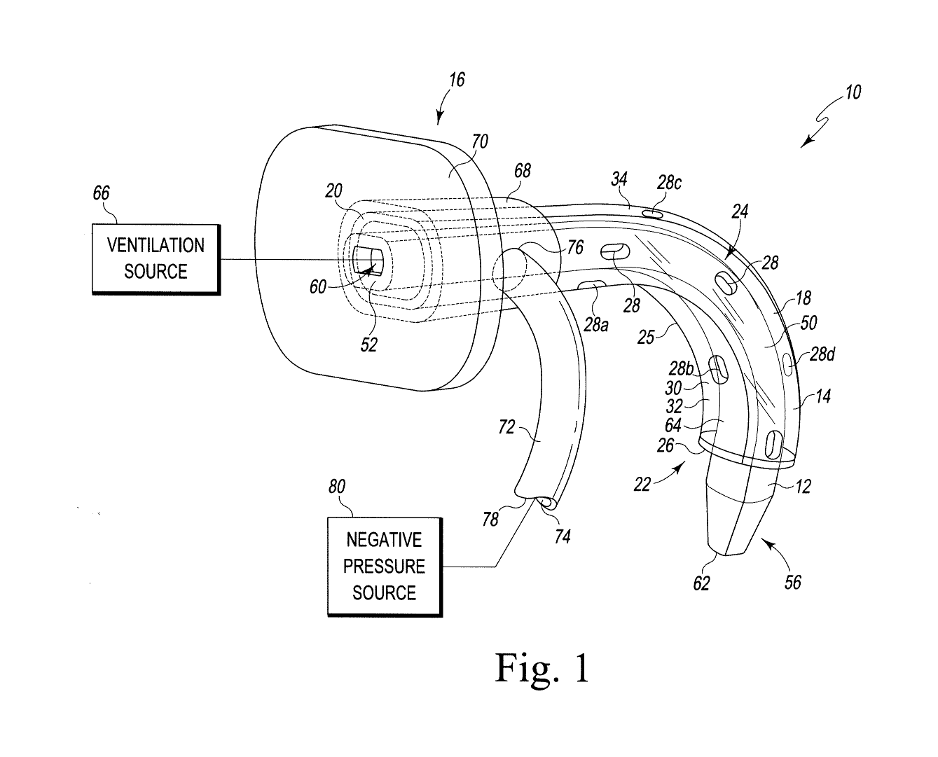

Referring now to FIG. 1, a medical instrument embodied as an oral suction device 10 (hereinafter device 10) is shown. The device 10 includes a curved inner tube 12, a curved outer tube 14 extending coaxially over part of the inner tube 12, and an end piece 16 secured to the tubes 12, 14. In the illustrative embodiment, the tubes 12, 14 and the end piece 16 are formed as a single integral component formed from semi-rigid polymer material. It will be appreciated that in other embodiments one or more components may be made separately and assembled together. It will also be appreciated that the device 10 may be formed silicon rubber or other material having a sufficient stiffness.

Each of the tubes 12, 14 has an oblong cross section. It will be appreciated that in other embodiments the cross sections of the tubes 12, 14 may be different. For example, the inner tube 12 may have a circular cross section and the outer tube 14 may have the oblong cross section shown in FIG. 1. As shown in FIG. 1, the tubes 12, 14 define an arc approximately ninety-degrees in length. It will be appreciated that in other embodiments the lengths of the tubes 12, 14 may extend a greater or lesser amount. Additionally, in other embodiments, the curvature of the tubes 12, 14 may also vary.

The outer tube 14 includes a body 18 extending from a proximal end 20 secured to the end piece 16 to a distal end 22. The body 18 has an internal chamber 24 defined by a sidewall 25 extending between the ends 20, 22. The chamber 24 is closed at the distal end 22 by an end wall 26 and is closed at the proximal end 20 by the end piece 16. As shown in FIG. 1, the chamber 24 has an oblong cross section to match the cross section of the tube 14. It will be appreciated that in other embodiments the cross section of the chamber may be circular, rectangular, or any other suitable shape. Additionally, the cross section of the chamber 24 may not match the cross section of the tube 14.

A number of holes 28 extend from the chamber 24 to the outer surface 30 of the outer tube 14. The number of holes 28 includes a first bottom hole 28a and a second bottom hole 28b extending from the chamber 24 through a bottom section 32 of the outer surface 30 and a first top hole 28c and a second top hole 28d extending from the chamber 24 through a top section 34 of the outer surface 30. It will be appreciated that in other embodiments the outer tube 14 may include additional or fewer holes 28 depending on the size and cross section of the outer tube 14. Additionally, the size and shape of the holes 28 may vary from that shown in FIG. 1.

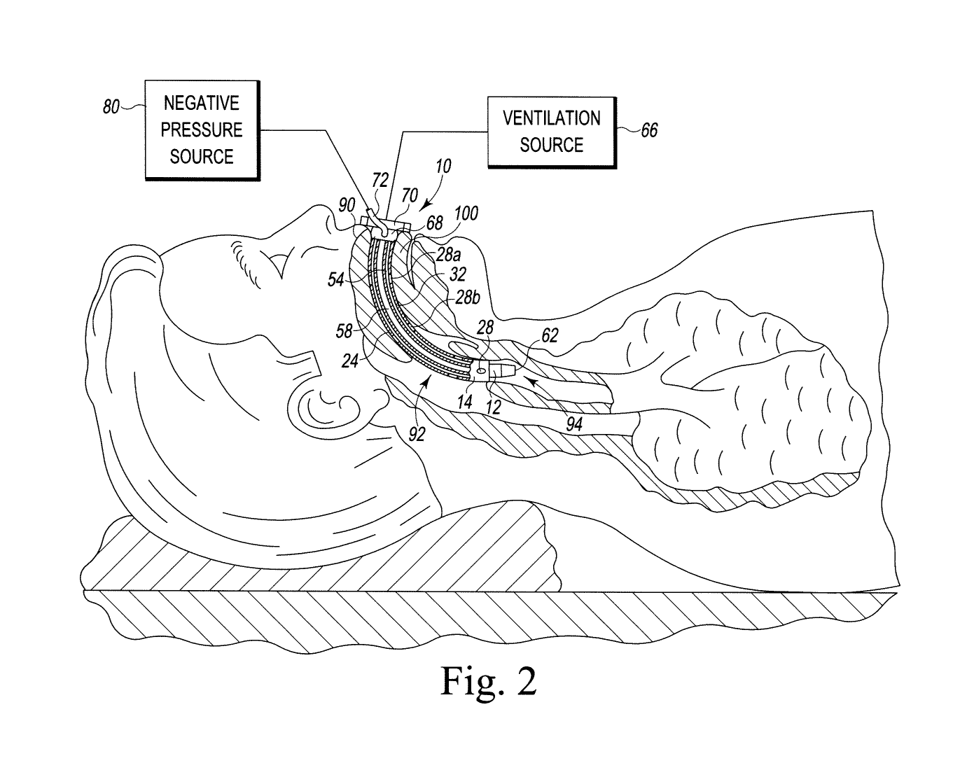

The inner tube 12 includes a body 50 extending from a proximal end 52. A section of the body 50 is positioned in the chamber 24 of the outer tube 14. The body 50 has a smaller cross section than the chamber 24 and is spaced apart from an inner surface 54 (see FIG. 2) of the tube 14. The body 50 extends through the end wall 26 of the tube 14 to a distal end 56 positioned beyond the end 22 of the outer tube 14. The distal end 56 of the tube 12 is tapered circumferentially to have a cone-shape. In other embodiments, the taper may be greater or less than that shown in FIG. 1. In still other embodiments, the taper of the distal end 56 may be removed.

The body 50 of the tube 12 has a passageway 58 defined therein. The passageway 58 extends from an opening 60 formed in the end piece 16 to another opening 62 formed in the distal end 56. A section 64 of the body 50 positioned in the chamber. 24 does not include any openings or holes along its length. In that way, the passageway 58 of the inner tube 12 is fluidly isolated from the chamber 24 of the outer tube 14.

As shown in FIG. 1, the opening 60 of the end piece 16 is coupled to a ventilation source 66. In some embodiments, the ventilation source 66 is a ventilator configured to advance gases through the passageway 58 and out the distal end 56 of the tube 12. In some embodiments, the ventilation source 66 may be an electromechanical ventilator or some other suitable ventilation device. Additionally, the ventilation source 66 may be a mask ventilator as is well known in the art. In some embodiments, the patient may not be connected to a ventilation source and may be allowed to ventilate spontaneously. The end piece 16 may include any fasteners, receivers, or other structures necessary to couple the opening 60 to the ventilation source 66.

The end piece 16 also has a block member 68 extending over the proximal end 20 of the outer tube 14 and a flange 70 extending outwardly from the block member 68. The block member 68 also has a catheter hose 72 secured thereto. The hose 72 has a passageway 74 defined therein, which extends from an end 76 fluidly coupled with the chamber 24 to another end 78 fluidly coupled to a negative pressure source 80. In that way, the chamber 24 and the hose 72 cooperate to place the holes 28 of the outer tube 14 in fluid communication with the negative pressure source 80. The hose 72 is formed from a plastic material having stiffness sufficient to avoid kinking and allow for continuous suction. The negative pressure source 80 is configured to provide continuous or intermittent suction.

While the hose 72 is shown formed integral with the outer tube 14, it will be appreciated that in other embodiments the outer tube 14 may include a nozzle or other connector such that the end 76 of the hose 72 may be removably coupled with the outer tube 14. In other embodiments, the device 10 may also include a control valve coupled to the end 78 of the hose 72. In such embodiments, the control valve is operable to selectively connect the negative pressure source 80 with the outer tube 14. The control valve may be operated manually or automatically via a computerized control system. In some embodiments, the control valve may be configured to move to the closed position when the ventilation source 66 applies positive pressure to the opening 60.

Referring now to FIG. 2, the device 10 is shown positioned in a patient. The flange 70 of the end piece 16 is engaged with the patient's lips 90 and is sized to discourage insertion of the flange 70 past the patient's lips 90. The block member 68 is positioned between the patient's upper teeth (not shown) and the patient's bottom teeth, thereby preventing the upper teeth from engaging the bottom teeth when the device 10 is positioned in the patient. The block member 68 blocks a patient's teeth from damaging a patient's tongue 100 or other devices inserted into the patient's mouth, such as, for example, an endotracheal tube or scope. The curvature of the tubes 12, 14 conforms to the curvature of a patient's airway, and the tubes 12, 14 extend downwardly from the patient's mouth into the lower airway 92. When positioned as shown in FIG. 2, the distal end 56 of the inner tube 12 is placed in the superglotic region 94 of the airway 92.

During inhalation, the ventilation source 66 advances oxygen and/or gases through the opening 60 of the end piece 16 and into the passageway 58 of the inner tube 12. The gas continues along the passageway 58 before being advanced out of the distal end 56. During exhalation, expelled gas is permitted to advance out of the patient's lungs and into the distal end 56 of the tube 12. Expelled gas then advances back up the passageway 58 and out through the opening 60 of the end piece 16.

The source 80 creates negative pressure within the hose 72 and the chamber 24, which is communicated to the plurality of holes 28 and creating suction along the length of the tube 14. The negative pressure is operable to remove, for example, any gastric contents and other unwanted materials from the patient's airway 92 in the area surrounding the outer tube 14. In that way, the risk of aspiration is reduced.

The negative pressure communicated to the plurality of holes 28 is also operable to draw the patient's tongue 100 into engagement with the outer tube 14. Specifically, the bottom holes 28a, 28b, apply negative pressure to a patient's tongue 100, thereby drawing the patient's tongue 100 into contact with the bottom section 32 of the outer surface 30 of the outer tube 14. Engagement with the outer tube 14 may prevent the patient's tongue 100 from blocking a portion of the patient's airway. As discussed above, the negative pressure source 80 may be configured to create continuous or intermittent suction. The operation of the negative pressure source 80 and the ventilation source 66 may be controlled via computerized control system such that the negative pressure source 80 is active during exhalation and inactive during inhalation such that movement of oxygen and other gases to the patient's lungs is not impaired.

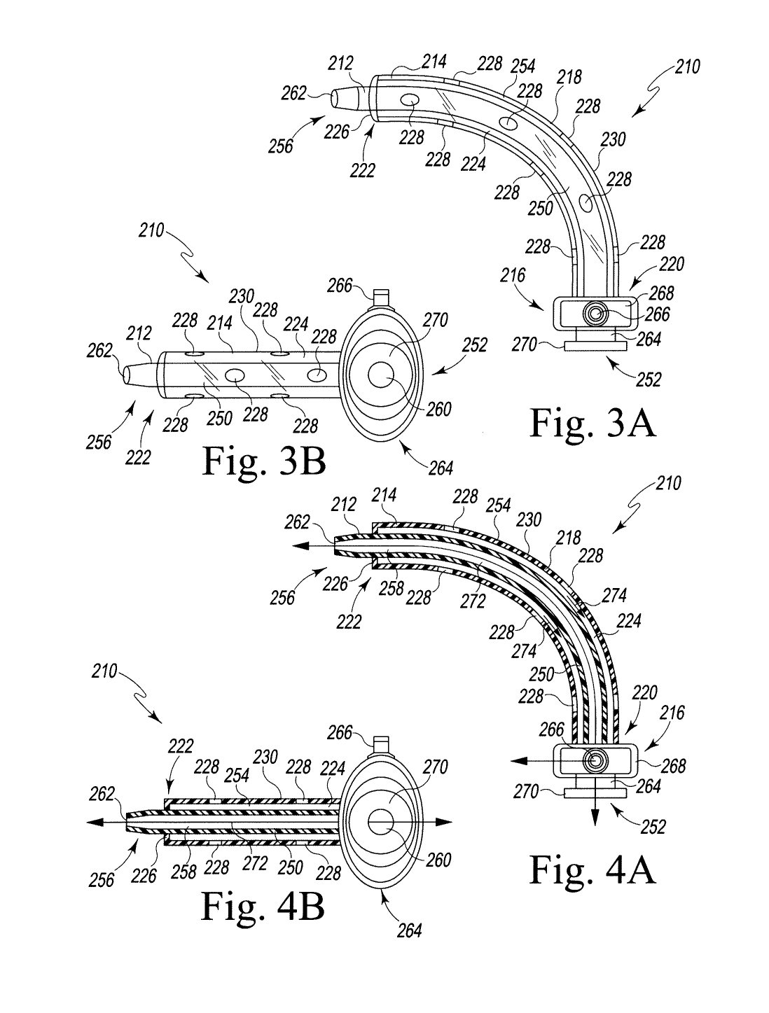

Another embodiment of an oral suction device similar to oral suction device 10 (hereinafter referred to as device 210) is shown in FIGS. 3 and 4. Some features of the embodiment illustrated in FIGS. 3 and 4 are substantially similar to those discussed above in reference to the embodiment of FIGS. 1 and 2. Such features in FIGS. 3 and 4 are designated with the same reference numbers as those used in FIGS. 1 and 2.

The device 210 includes a curved inner tube 212, a curved outer tube 214 extending coaxially over part of the inner tube 212, and an end piece 216 secured to the tubes 212, 214. As shown in FIGS. 3 and 4, each of the tubes 212, 214 has a circular cross section. The outer tube 214 includes a body 218 extending from a proximal end 220 secured to the end piece 216 to a distal end 222. The body 218 has a passageway or chamber 224 defined therein extending between the ends 220, 222. The chamber 224 is closed at the distal end 222 by an end wall 226 and is closed at the proximal end 220 by the end piece 216. A number of holes 228 extend from the chamber 224 to the outer surface 230 of the outer tube 14. The holes 228 are positioned over the entire circumference of the outer surface 230.

The inner tube 212 includes a body 250 extending from a proximal end 252 positioned in the chamber 224 of the outer tube 214. The body 250 has a smaller cross section than the chamber 224 and is spaced apart from the inner surface 254 of the tube 214. The body 250 extends through the end wall 226 of the tube 214 to a distal end 256 positioned beyond the end 222 of the outer tube 214. The distal end 256 of the tube 12 is tapered circumferentially to have a cone-shape. The body 250 of the tube 212 has a passageway 258 defined therein. The passageway 258 extends from an opening 260 formed in the end piece 216 to another opening 262 formed in the distal end 256. The opening 260, like the opening 60 of the device 10 of FIGS. 1 and 2, is configured to be coupled to a ventilation source (not shown).

The end piece 216 includes a block member 268 extending over the proximal end 220 of the outer tube 14. The block member 268 includes an oval-shaped body 264 sized to be positioned between the patient's upper teeth and the patient's bottom teeth. As shown in FIG. 3B, the body 264 has a larger cross section that the body 218 of the tube 214. As a result, the patient's upper teeth and bottom teeth engage with the body 264 of the block member 268 rather than the outer surface 230 of the tube 214. A nozzle 266 is secured to a side 269 of the body 264 of the block member 268. The nozzle 266 is sized to receive an end of a hose coupled to a negative pressure source (not shown).

The end piece 216 also includes a flange 270 spaced apart from the block member 268. The flange 270 has a circular cross section (see FIG. 3B) that is smaller than the cross section of the block member 268. In that way, the device 210 may be positioned in the patient with the flange 270 located inside or outside of the patient's mouth.

Referring now to FIGS. 4A and 4B, arrows 272, 274 indicate the flow of gases and fluids through the device 210. When the device 210 is coupled to a ventilation source, gas is permitted to advance back and forth along the passageway 258 into and out of the patient's lungs along the path indicated by arrow 272. When the device 210 is connected to a negative pressure source via the nozzle 266, fluids and/or gases may be suctioned out of the patient's airway through the holes 228 and along the chamber 224 in the direction indicated by arrow 274. The flow of gases and fluids through the chamber 224 and the passageway 258 may occur concurrently or consecutively depending on the needs of the patient and the nature of the medical procedure.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. An oral suction device for insertion into an airway of a patient comprising:
a first tube having a first gas port configured to be coupled to a ventilation source; and
a second tube extending coaxially over at least a first section of the first tube, the second tube including (i) a second gas port configured to be coupled to a negative pressure source and (ii) an outer surface having at least one hole defined therein.

2. The oral suction device of claim 1, wherein the second gas port is located at a proximal end of the second tube.

3. The oral suction device of claim 1, wherein the outer surface includes a top section having a first hole defined therein.

4. The oral suction device of claim 3, wherein the outer surface includes a bottom section opposite the top section, the bottom section having a second hole defined therein and being configured to face the patient's tongue when the oral suction device is inserted into the airway of a patient.

5. The oral suction device of claim 4, wherein the second hole is sized to permit air from the second tube to pull the patient's tongue into contact with the outer surface of the second tube when negative pressure is introduced into the second tube through the second gas port.

6. The oral suction device of claim 5, further comprising an end piece extending outward from a proximal end of the second tube.

7. The oral suction device of claim 6, wherein the end piece includes (i) a block member extending outwardly from the second tube, and (ii) a flange extending outwardly from a proximal end of the block member.

8. The oral suction device of claim 7, further comprising a hose coupled to the second gas port and extending outwardly from the block member.

9. The oral suction device of claim 7, wherein the flange extends outwardly from a proximal end of the first tube.

10. The oral suction device of claim 7, wherein the flange is sized to prevent passage of the flange past the lips of the patient when the oral suction device is inserted in the airway of the patient.

11. The oral suction device of claim 6, further comprising an end wall extending inwardly from a distal end of the second tube to the first tube.

12. The oral suction device of claim 11, wherein a second section of the first tube extends outwardly from the end wall.

13. The oral suction device of claim 12, wherein the second section of the first tube is tapered.

14. The oral suction device of claim 5, wherein the first tube and the second tube are curved to conform to the airway of the patient when the oral suction device is inserted into the airway of the patient.

15. The oral suction device of claim 14, wherein the first tube is a predetermined length such that a distal end of the first tube is positioned in a superglotic region of the airway of the patient when the oral suction device is inserted in the airway of the patient.

16. An oral suction device for insertion into an airway of a patient comprising:
a first tube having a first gas port configured to be coupled to a ventilation source and
a second tube coupled to the first tube, the second tube having a chamber defined therein and a second gas port configured to be coupled to a negative pressure source,
wherein (i) a first section of the first tube is positioned in the chamber of the second tube, and (ii) the second tube includes at least one hole extending between the chamber and an outer surface of the second tube.

17. The oral suction device of claim 16, wherein a second section of the first tube extends out of the chamber.

18. The oral suction device of claim 17, wherein the first section of the first tube includes a proximal end of the first tube and the second section of the first tube includes a distal end of the first tube.

19. The oral suction device of claim 18, wherein the distal end of the first tube is tapered around a circumference of the first tube.

20. The oral suction device of claim 17, further comprising an end wall extending between a distal end of the second tube and an outer surface of the first tube to enclose a distal end of the chamber.

21. The oral suction device of claim 20, wherein a distal end of the first tube extends from the end wall.

22. The oral suction device of claim 16, further comprising an end piece connecting the first tube and the second tube to enclose a proximal end of the chamber.

23. The oral suction device of claim 22, wherein the end piece is configured to engage a patient's lips and sized to prevent the end piece from passing beyond the patient's lips when the oral suction device is inserted in the airway of the patient.

24. The oral suction device of claim 23, wherein the end piece includes (i) a block member extending outwardly from the second tube, and (ii) a flange extending outwardly from a proximal end of the block member.

25. The oral suction device of claim 16, wherein the second tube has an oblong cross section such that the chamber has an oblong cross section.

26. The oral suction device of claim 25, wherein the second tube is curved such that a distal end of the chamber is angled approximately ninety degrees from a proximal end of the chamber.

27. The oral suction device of claim 26, wherein the first tube is curved such that a distal end of the first tube is angled approximately ninety degrees from a proximal end of the first tube.

28. The oral suction device of claim 25, wherein the second tube includes a bottom section having at least one hole extending between the chamber and the outer surface of the second tube, the bottom section being configured to face the patient's tongue when the oral suction device is inserted into the airway of the patient.

29. The oral suction device of claim 28, wherein the second tube includes a top section opposite the bottom section, the top section having at least one hole extending between the chamber and the outer surface of the second tube.

30. The oral suction device of claim 29, wherein the second tube includes a side section connecting the bottom section and the top section, the side section having at least one hole extending between the chamber and the outer surface of the second tube.

31. A method of providing suction to an airway of a patient comprising
inserting an oral suction device into a patient's airway, the oral suction device including (i) a first tube having a first gas port configured to be coupled to a ventilation source; and (ii) a second tube extending coaxially over at least a first section of the first tube, the second tube configured to be coupled to a negative pressure source,
applying ventilation to first tube of the oral suction device, and
applying vacuum pressure to the second tube of the oral suction device.

32. The method of claim 31, wherein applying vacuum pressure to the second tube of the oral suction device includes drawing the patient's tongue into contact with the second tube by passing vacuum pressure through a first hole formed through a bottom side of the second tube.

33. The method of claim 32, wherein applying vacuum pressure to the second tube of the oral suction device includes removing unwanted materials from the patient's mouth by passing vacuum pressure through a second hole formed through a second side of the second tube.

34. The method of claim 33, wherein applying ventilation to the first tube of the oral suction device includes (i) applying positive ventilation pressure to the first tube and (ii) applying negative ventilation pressure to the first tube, applying vacuum pressure to the second tube only being performed when negative ventilation pressure is applied to the first tube.

35. The method of claim 31, wherein inserting the oral suction device into a patient's airway includes positioning a block member between the patient's upper teeth and the patient's bottom teeth, the block member extending outwardly from the second tube.

36. The method of claim 31, wherein inserting the oral suction device into a patient's airway includes positioning a flange in contact with the patient's lips, the flange extending outwardly from a proximal end of the block member and being sized to discourage the flange from being passed beyond the patient's lips.
